(19) United States
(12) Patent Application Publication
(10) Pub. No.: US 2025/0127739 A1
(43) Pub. Date: Apr. 24, 2025

(54) STABLE GABAPENTIN POWDER

(71) Applicant: Avion Phamaceuticals, LLC, Alpharetta, GA (US)

(72) Inventors: Samuel A. TESTINO, Alpharetta, GA (US); Dongwei GUO, Alpharetta, GA (US); Benjamin BURGESS, Alpharetta, GA (US)

(21) Appl. No.: 18/685,701

(22) PCT Filed: Aug. 30, 2022
(86) PCT No.: PCT/US2022/041994

§ 371 Date: Feb. 22, 2024

Related U.S. Application Data
(60) Provisional application No. 63/238,870, filed on Aug. 31, 2021.

(51) Int. Cl. A61K 31/195; A61K 9/16; A61K 47/22

(52) U.S. Cl. CPC A61K 31/195; A61K 9/1623; A61K 47/22

(57) ABSTRACT

Described are room temperature stable gabapentin powders that can be readily reconstituted in water for dosing as an oral solution. The gabapentin powder formulations have improved chemical stability, palatability, portability, storage, and ease of administration, as well as fast reconstitution time, and prolonged product shelf life. The gabapentin powder formulations comprise gabapentin, a sugar alcohol, one or more artificial sweeteners and, optionally, a flavoring agent.

### RELATED APPLICATION INFORMATION

This application claims priority to U.S. Application No. 63/238,870 filed on Aug. 31, 2021, the contents of which are herein incorporated by reference.

### TECHNICAL FIELD

The present disclosure relates to room temperature stable gabapentin powder formulations.

### BACKGROUND

Gabapentin and its pharmaceutically acceptable salts are currently used for treating and improving a variety of neurological and cerebral conditions, such as epilepsy, seizures, fainting attacks, hypokinesis, neuropathic pain, chronic pain, and cranial traumas. However, aqueous gabapentin is difficult to formulate due to formation of an intramolecular lactam derivative via intramolecular cyclization. Due to this instability of gabapentin, most current gabapentin solution products must be stored under refrigerated conditions, which is non-portable and inconvenient.

### SUMMARY OF THE INVENTION

Described herein are powder compositions of gabapentin characterized by increased chemical stability and improved storage requirements for reconstitution in aqueous formulations.

Also described herein are unit dosage packs comprising a powder formulation of gabapentin.

### DETAILED DESCRIPTION

Disclosed herein are gabapentin powder formulations for reconstitution in aqueous formulations which have improved chemical stability, palatability, portability, storage, and ease of administration, as well as fast reconstitution time, and prolonged product shelf life. The gabapentin powder formulations comprise gabapentin, a sugar alcohol (e.g., erythritol), one or more artificial sweeteners, and, optionally, a flavoring agent. No additional preservative was required for the improved stability. The product was easily reconstituted in water and possessed enhanced chemical stability when stored at room temperature and more accelerated conditions, contrary to currently available gabapentin formulations.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “gabapentin” as used in the invention refers to 1-(aminomethyl)-1-cyclohexane acetic acid or its pharmaceutically acceptable salt(s), hydrate(s), solvate(s) and physiologically functional derivative(s) and precursors thereof.

As used herein, the terms “administering,” “providing,” and “introducing” are used interchangeably herein and refer to the placement of the compositions of the disclosure into a subject by a method or route which results in at least partial localization a desired site. The compounds or compositions can be administered by any appropriate route which results in delivery to a desired location in the subject.

As used herein, the term “preventing” refers to partially or completely delaying onset of a disease, disorder and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

As used herein, “treat,” “treating,” and the like means a slowing, stopping, or reversing of progression of a disease or disorder when provided a composition described herein to an appropriate control subject. The term also means a reversing of the progression of such a disease or disorder. As such, “treating” means an application or administration of the methods, compounds, or compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

### 2. Powder Formulations

In one aspect disclosed is powdered formulations of gabapentin comprising: gabapentin, a sugar alcohol, and one or more artificial sweeteners, wherein the formulations do not comprise any additional preservatives.

As used herein, the term “sugar alcohol” is defined as a polyhydric alcohol (also known as a polyol) formed by the reduction of the carbonyl group of a sugar to a hydroxyl group, with no more than one hydroxy group being attached to any one carbon atom of the sugar alcohol. Exemplary sugar alcohols include, but are not limited to: adonitol or ribitol; allitol; altritol (D-altritol, L-altritol, and D, L altritol); arabinitol (D-arabinitol, L-arabinitol, and D, L arabinitol); dulcitol (a.k.a galactitol); erythritol; galaxitol; glucitol (D-glucitol, L-glucitol, and D, L glucitol); iditol (D-iditol and L-iditol); inositol; isomalt; lactitol; maltitol; mannitol (D-mannitol, L-mannitol, and D, L mannitol); perseitol; rhamnitol; sorbitol; threitol (D-threitol, L-threitol, and D, L threitol); and xylitol. Any mixture of the described sugar alcohols may be employed in the disclosed formulations. In some embodiments, the sugar alcohol is selected from the group consisting of: erythritol, sorbitol, xylitol, mannitol, galactitol, threitol, and combinations thereof. In select embodiments, the sugar alcohol comprises erythritol.

As used herein, the term “artificial sweetener” includes synthetic sugar substitutes derived from naturally occurring substances, such as herbs or sugar itself, or chemically synthesized synthetic sugar substitutes. Exemplary artificial sweeteners include, but are not limited to: aspartame; advantame; neotame; alitame; sodium saccharin; calcium saccharin; acesulfame potassium; sodium cyclamate; calcium cyclamate; neohesperidin dihydrochalcone; steviol glycosides; and sucralose. In some embodiments, the one or more artificial sweeteners are selected from the group consisting of: aspartame, sucralose, neotame, acesulfame potassium, saccharin, advantame, and combinations thereof. In select embodiments, the one or more artificial sweeteners comprise acesulfame potassium and saccharin sodium.

The formulation may comprise the gabapentin and the sugar alcohol at a weight percent ratio of 1:2 to 1:5. In some embodiments, the formulation comprises the gabapentin and the sugar alcohol at a weight percent ratio of about 1:2, about 1:2.5, about 1:3, about 1:3.5, about 1:4, about 1:4.5, or about 1:5. In select embodiments, the formulation comprises the gabapentin and the sugar alcohol at a weight percent ratio of about 1:2 to about 1:4. In certain embodiments, the formulation comprises the gabapentin and the sugar alcohol at a weight percent ratio of about 1:3 to about 1:4.

In some embodiments, the formulation comprises the gabapentin and the one or more artificial sweeteners at a weight percent ratio of 1:0.01 to 1:0.5. In some embodiments, the formulation comprises the gabapentin and the one or more artificial sweeteners at a weight percent ratio of about 1:0.02, about 1:0.05, about 1:0.1, about 1:0.2, about 1:0.3, or about 1:0.4. In select embodiments, the formulation comprises the gabapentin and the one or more artificial sweeteners at a weight percent ratio of about 1:0.2 to about 1:0.3.

The formulation may comprise 10-30 weight percent gabapentin. In some embodiments, the formulation comprises 10-25 weight percent gabapentin, 10-20 weight percent gabapentin, 10-15 weight percent gabapentin, 15-30 weight percent gabapentin, 15-25 weight percent gabapentin, 15-20 weight percent gabapentin, 20-30 weight percent gabapentin, 20-25 weight percent gabapentin, or 25-30 weight percent gabapentin.

In select embodiments, the formulation comprises about 10 wt % gabapentin, about 11 wt % gabapentin, about 12 wt % gabapentin, about 13 wt % gabapentin, about 14 wt % gabapentin, about 15 wt % gabapentin, about 16 wt % gabapentin, about 17 wt % gabapentin, about 18 wt % gabapentin, about 19 wt % gabapentin, about 20 wt % gabapentin, about 21 wt % gabapentin, about 22 wt % gabapentin, about 23 wt % gabapentin, about 24 wt % gabapentin, about 25 wt % gabapentin, about 26 wt % gabapentin, about 27 wt % gabapentin, about 28 wt % gabapentin, about 29 wt % gabapentin, or about 30 wt % gabapentin.

The formulation may comprise 60-80 weight percent of the sugar alcohol. In some embodiments, the formulation comprises 60-75 weight percent of the sugar alcohol, 60-70 weight percent of the sugar alcohol, 60-65 weight percent of the sugar alcohol, 65-80 weight percent of the sugar alcohol, 65-75 weight percent of the sugar alcohol, 65-70 weight percent of the sugar alcohol, 70-80 weight percent of the sugar alcohol, 70-75 weight percent of the sugar alcohol, or 78-80 weight percent of the sugar alcohol.

In select embodiments, the formulation comprises about 60 wt % sugar alcohol, about 61 wt % sugar alcohol, about 62 wt % sugar alcohol, about 63 wt % sugar alcohol, about 64 wt % sugar alcohol, about 65 wt % sugar alcohol, about 66 wt % sugar alcohol, about 67 wt % sugar alcohol, about 68 wt % sugar alcohol, about 69 wt % sugar alcohol, about 70 wt % sugar alcohol, about 71 wt % sugar alcohol, about 72 wt % sugar alcohol, about 73 wt % sugar alcohol, about 74 wt % sugar alcohol, about 75 wt % sugar alcohol, about 76 wt % sugar alcohol, about 77 wt % sugar alcohol, about 78 wt % sugar alcohol, about 79 wt % sugar alcohol, or about 80 wt % sugar alcohol.

The formulation may comprise 0.2-10% of one or more artificial sweeteners. In some embodiments, the formulation comprises 0.2-9% of one or more artificial sweeteners, 0.2-8% of the one or more artificial sweeteners, 0.2-7% of one or more artificial sweeteners, 0.2-6% of one or more artificial sweeteners, 0.2-5% of one or more artificial sweeteners, 0.2-4% of one or more artificial sweeteners, 0.2-3% of one or more artificial sweeteners, 0.2-2% of one or more artificial sweeteners, 0.2-1% of one or more artificial sweeteners, 0.2-0.5% of one or more artificial sweeteners, 0.5-9% of one or more artificial sweeteners, 0.5-8% of one or more artificial sweeteners, 0.5-7% of one or more artificial sweeteners, 0.5-6% of one or more artificial sweeteners, 0.5-5% of one or more artificial sweeteners, 0.5-4% of one or more artificial sweeteners, 0.5-3% of one or more artificial sweeteners, 0.5-2% of one or more artificial sweeteners, 0.5-1% of one or more artificial sweeteners, 1-9% of one or more artificial sweeteners, 1-8% of one or more artificial sweeteners, 1-7% of one or more artificial sweeteners, 1-6% of one or more artificial sweeteners, 1-5% of one or more artificial sweeteners, 1-4% of one or more artificial sweeteners, 1-3% of one or more artificial sweeteners, 1-2% of one or more artificial sweeteners, 2-9% of one or more artificial sweeteners, 2-8% of one or more artificial sweeteners, 2-7% of one or more artificial sweeteners, 2-6% of one or more artificial sweeteners, 2-5% of one or more artificial sweeteners, 2-4% of one or more artificial sweeteners, 2-3% of one or more artificial sweeteners, 3-9% of one or more artificial sweeteners, 3-8% of one or more artificial sweeteners, 3-7% of one or more artificial sweeteners, 3-6% of one or more artificial sweeteners, 3-5% of one or more artificial sweeteners, 3-4% of one or more artificial sweeteners, 4-9% of one or more artificial sweeteners, 4-8% of one or more artificial sweeteners, 4-7% of one or more artificial sweeteners, 4-6% of one or more artificial sweeteners, 4-5% of one or more artificial sweeteners, 5-9% of one or more artificial sweeteners, 5-8% of one or more artificial sweeteners, 5-7% of one or more artificial sweeteners, 5-6% of one or more artificial sweeteners, 6-9% of one or more artificial sweeteners, 6-8% of one or more artificial sweeteners, 6-7% of one or more artificial sweeteners, 7-9% of one or more artificial sweeteners, 7-8% of one or more artificial sweeteners, or 8-9% of one or more artificial sweeteners.

In select embodiments, the powder formulation comprises 10-30% by weight gabapentin, 60-80% by weight sugar alcohol, and 0.2-10% by weight one or more artificial sweeteners. In certain embodiments, the powder formulation comprises 15-25% by weight gabapentin, 70-80% by weight sugar alcohol, and 2-6% by weight one or more artificial sweeteners. In certain embodiments, the powder formulation comprises about 20% by weight gabapentin, about 75% by weight sugar alcohol (e.g., erythritol), and about 4-6% by weight one or more artificial sweeteners (e.g., acesulfame potassium and saccharin sodium).

In some embodiments, the powder formulation further comprises a flavoring agent. Suitable flavors include menthol, mint (e.g., natural mint, peppermint), cream, and fruit (e.g., raspberry, strawberry, cheery) flavoring agents. The amount of flavoring agent(s), when used, is typically about 0.1 to about 1.0%. In some embodiments, the powder formulation comprises less than 1% by weight flavoring agent (e.g., about 0.1%, about 0.2%, about 0.3%, about 0.4%, about 0.5%, about 0.6%, about 0.7%, about 0.8%, about 0.9%, about 1%).

In certain embodiments, the powder formulation comprises about 20% by weight gabapentin, about 75% by weight sugar alcohol (e.g., erythritol), about 4-6% by weight one or more artificial sweeteners (e.g., acesulfame potassium and saccharin sodium), and 0.1-1% flavoring agent.

The powder formulation may further comprise an excipient. The term “pharmaceutically acceptable carrier,” as used herein, means a non-toxic, inert solid filler, diluent, or bulking agent auxiliary of any type. Some example of materials which can serve as excipients include starches such as, but not limited to, corn starch and potato starch; cellulose and its derivatives such as, but not limited to, sodium carboxymethyl cellulose, ethyl cellulose and cellulose acetate; cyclodextrins, or other synthetic polymers.

Gabapentin is known to chemically degrade to a more toxic gabapentin lactam form under the influence of various stress conditions such as heat and moisture. The combination of components disclosed herein control the generation of gabapentin lactam impurities in the powder formulation over time.

In the disclosed formulation, gabapentin lactam impurities are well controlled. The gabapentin lactam impurity may be below 0.05% (e.g., 0.04%, 0.03%, 0.02%, 0.01%, or less) when stored at room temperature or 25° C. with 60% relative humidity for at least 3 months (e.g., at least 2 weeks, at least 4 weeks, at least 6 weeks, at least 2 months, at least 3 months, at least 4 months, at least 5 months, at least 6 months, or more). The gabapentin lactam impurity may be below 0.5% (e.g., 0.4%, 0.3%, 0.2%, 0.1%, or less) at an accelerated condition of at least 4 weeks (e.g., at least 6 weeks, at least 2 months, at least 3 months, or more) at 40° C. and 75% relative humidity (RH). In some embodiments, the gabapentin lactam impurity is below 0.5% at an accelerated condition of at least 6 months at 40° C. and 75% RH. In some embodiments, the gabapentin lactam impurity is below 1% following at least 4 weeks at 60° C. In other embodiments, the gabapentin lactam impurity is below 0.2% following at least 12 months at 25° C. and 60% relative humidity (RH). In still other embodiments, the gabapentin lactam impurity is below 0.2% following at least 18 months at 25° C. and 60% relative humidity (RH).

The disclosed formulations exhibit good dissolution times. The powder formulation disclosed herein may exhibit a dissolution time of less than 1 minute when dissolved at 1-2% weight to volume of a liquid (e.g., water) at room temperature. In some embodiments, the dissolution time is less than 50 seconds, less than 40 second, less than 30 seconds, less than 20 seconds or less than 10 second when the powdered formulation is dissolved in 1-2% weight to volume of a liquid (e.g., water).

The disclosure further provides a method of making the disclosed powder formulations comprising direct powder mixing of the gabapentin, the sugar alcohol, the one or more artificial sweeteners, and, when used, the flavoring agent.

In some embodiments, the method further comprises sieving the gabapentin, the sugar alcohol, the one or more artificial sweeteners, and, when used, the flavoring agent prior to mixing. In select embodiments, gabapentin, the sugar alcohol, and, when used, the flavoring agent are sieved using a smaller size sieve than that of the one or more artificial sweeteners.

### 3. Unit Dose

The disclosure further provides a unit dose comprising a powder formulation as described herein. A “unit dose” or “unit dosage” as used herein means an amount or dose of a formulation as described herein prepared in an individual packet or container for convenience, or safety. Unit doses include any single dose container, such as individual vials, sachets, packets, stick packs, tablets, capsules, pods, and the like.

A “stick pack” as used herein means a flexible disposable or single use container comprising a unit dosage of the disclosed formulations. In some embodiments, the container is plastic, paper, aluminum, mylar, or thermoplastic polymer resin.

In some embodiments, the powder formulation, is formulated into a unit dose of between about 0.5 g and about 10 g total. In some embodiments, the unit dose comprises about 0.5 g, about 1.0 g, about 1.5 g, about 2.0 g, about 2.5 g, about 3.0 g, about 3.5 g, about 4.0 g, about 4.5 g, about 5.0 g, about 5.5 g, about 6.0 g, about 6.5 g, about 7.0 g, about 7.5 g, about 8.0 g, about 8.5 g, about 9.0 g, about 9.5 g, or about 10 g.

### 4. Methods of Use

The disclosed powder formulations may be used in methods of treating a disease or disorder in a subject in need thereof. The method may comprise administering an effective amount of the powder formulation to the subject in need thereof. In some embodiments, the method further comprises dissolving the powder formulation in a volume of water prior to administration.

In some embodiments, the administering comprises oral administration. In some embodiments, the administering comprises enteral feeding. In the context of this application, “enteral feeding” refers to the act of being administered the disclosed powder formulations with a feeding tube or other enteral feeding devices known in the art. Generally, enteral feeding is administered to patients in hospitals, in nursing homes and to subjects in the context of home care.

Disorders in which a patient would benefit from treatment with the formulations disclosed herein include neurological or central nervous system diseases or conditions. In some embodiments, the disease or disorder comprises seizures, neuropathic pain, chronic pain, neurodegenerative disorders, hypokinesia, and bradykinesia. For example, the disclosed formulation can be used to treat or prevent neuropathic pain, caused by herpes virus or shingles (herpes zoster virus). The disclosed formulation can also be used to treat, prevent, or lessen the severity of seizures, convulsions, or epilepsy.

In the methods of use described herein, additional therapeutic agent(s) or second therapies may be administered simultaneously or sequentially with the disclosed formulations. Sequential administration includes administration before or after the disclosed formulations. In other embodiments, there may be an interval of time between administration of the additional therapeutic agent and the disclosed formulations

### 5. Examples

### Example 1

### Assay for Gabapentin and Gabapentin Lactam

An analytical HPLC method for assaying gabapentin and gabapentin lactam in powder formulation product was developed based on a USP method. The detailed parameters of the HPLC conditions are shown in Table 1. Typical HPLC chromatograms indicate in an early peak at about 11-12 minutes gabapentin, while the lactam elutes much later at 44-45 minutes.

TABLE 1
HPLC Conditions for Assay and Impurity Analysis
Column: Phenomenex Luna C-8(2), 240 ×
4.6 mm, 10 μm, 100 Å, P/N: 00G-4250-E0

| | |
|---|---|
| Mobile Phase A: 0.0088M | Mobile Phase A: 0.0088M Monobasic |
| Monobasic Potassium | Potassium Phosphate in 60:40 ACN:water, |
| Phosphate in Water, pH = 6.9 | pH = 6.9 |
| Wavelength: 210 nm | Injection Volume: 10 μL |
| Column Temperature: 60° C. | Sample Temperature: 25° C. |
| Needle Wash: NA | Run Time: 53 minutes |

Gradient Elution Table

| Time (min) | Flow Rate (mL/min) | % A | % B |
|---|---|---|---|
| 0 | 0.70 | 90 | 10 |
| 7 | 0.70 | 90 | 10 |
| 48 | 0.70 | 50 | 50 |
| 48.1 | 0.70 | 90 | 10 |
| 53 | 0.70 | 90 | 10 |

### Example 2

### Dried Powder Formulations with CMC-Na

Dried powder formulations were developed based on an approved product solution formulations containing CMC-Na, methylparaben, propylparaben, acesulfame potassium, and saccharin sodium. Formulations in Table 2 are prepared at around 76% drug loading by wet granulation and samples were dried in oven at 40° C. for 2 hours.

TABLE 2
Wet Granulation Powder Formulations

| | Amount (g) | | | |
|---|---|---|---|---|
| Ingredients | 1108-P1 | 1108-P2 | 1108-P3 | 1108-P4 |
| Gabapentin | 5 | 5 | 5 | 5 |
| CMC-NA-7L(M)F | 1 (LF) | 1 (MF) | 1 (LF) | 1 (LF) |
| Methylparaben | 0.12 | 0.12 | 0.08 | 0.12 |
| Propylparaben | 0.06 | 0.06 | 0.04 | 0.06 |
| Acesulfame | 0.1 | 0.1 | 0.1 | 0.1 |
| Potassium | | | | |
| Saccharin | 0.1 | 0.1 | 0.1 | 0.1 |
| Sodium | | | | |
| PBS (0.1M) | 1.2 mL | 1.2 mL | 1.2 mL | 1.2 mL |
| | (pH = 6.1) | (pH = 6.1) | (pH = 6.1) | (pH = 6.8) |

Short-term stability of the dried powder formulations was evaluated under accelerated storage conditions. Powder formulations were more stable at 40° C./75% RH than at 60° C. and formulations containing CMC-Na-7LF presented lower lactam levels at both conditions (Table 3). The formulation at pH 6.8 proved to be the most stable.

TABLE 3
Short-term Stability

| | | Loading | Purity | Lactam |
|---|---|---|---|---|
| Formulation | Condition | (%) | (%) | (%) |
| 1108-P1 | T0 | 74.0 | 99.98 | 0.02 |
| | 1 week @ 60° C. | 76.0 | 99.72 | 0.28 |
| | 2 weeks @ 60° C. | 75.3 | 99.72 | 0.28 |
| | 3 weeks @ 60° C. | 75.9 | 99.46 | 0.54 |
| | 4 weeks @ 60° C. | 76.2 | 99.37 | 0.63 |
| | 4 weeks @ 40° C./RH75% | 75.3 | 99.81 | 0.19 |
| | 8 weeks @ 40° C./RH75% | 77.3 | 99.61 | 0.39 |
| 1108-P2 | T0 | 76.3 | 99.98 | 0.02 |
| | 1 week @ 60° C. | 77.3 | 99.75 | 0.25 |
| | 2 weeks @ 60° C. | 76.8 | 99.61 | 0.39 |
| | 3 weeks @ 60° C. | 78.8 | 99.46 | 0.54 |
| | 4 weeks @ 60° C. | 77.7 | 99.27 | 0.73 |
| | 4 weeks @ 40° C./RH75% | 76.8 | 99.80 | 0.20 |
| | 8 weeks @ 40° C./RH75% | 76.3 | 99.59 | 0.41 |
| 1108-P3 | T0 | 78.1 | 99.98 | 0.02 |
| | 1 week @ 60° C. | 79.4 | 99.83 | 0.17 |
| | 2 weeks @ 60° C. | 80.3 | 99.74 | 0.26 |
| | 3 weeks @ 60° C. | 79.3 | 99.61 | 0.39 |
| | 4 weeks @ 60° C. | 80.6 | 99.51 | 0.49 |
| | 4 weeks @ 40° C./RH75% | 80.3 | 99.81 | 0.19 |
| | 8 weeks @ 40° C./RH75% | 80.9 | 99.66 | 0.34 |
| 1108-P4 | T0 | 77.8 | 99.98 | 0.02 |
| | 1 week @ 60° C. | 78.1 | 99.89 | 0.11 |
| | 2 weeks @ 60° C. | 78.2 | 99.83 | 0.17 |
| | 3 weeks @ 60° C. | 78.0 | 99.73 | 0.27 |
| | 4 weeks @ 60° C. | 79.7 | 99.62 | 0.38 |
| | 4 weeks @ 40° C./RH75% | 76.4 | 99.90 | 0.10 |
| | 8 weeks @ 40° C./RH75% | 78.8 | 99.86 | 0.14 |

To improve the ease of dosing and manufacture, a single dose formulation was developed with decreased drug loading and lack of preservatives. A set of formulations with different flavors were prepared by wet granulation with 20% drug loading as presented in Table 4 and three different flavors were further evaluated.

TABLE 4
Wet Granulation Formulation with Decreased Drug Loading

| Ingredients | Amount (g) |
|---|---|
| Gabapentin | 3.0 |
| CMC-NA-7L(M)F | 6.5 |
| Acesulfame Potassium | 0.5 |
| Saccharin Sodium | 0.5 |
| Flavor | 0.5 |
| PBS (0.1M) | 2.0 mL |
| | (pH = 6.8) |

Powder formulations were stored at accelerated conditions (40° C./75% RH and 60° C.) for short-term stability screening and evaluation of purity and lactam levels. The formulations resulted in 0.25-0.60% lactam after 4 weeks stored at 40° C./75% RH.

Although formulations were stable at accelerated conditions, the existence of CMC-Na-7LF caused slow dissolution due to the high viscosity of the polymer; the reconstitution time was greater than 60 seconds in room temperature water.

### Example 3

### Excipient-Gabapentin Compatibility

To evaluate the excipient suitability and compatibility, a variety of different excipients including PVP-VA64, PVP-K30, poloxamer 188, poloxamer 407, MCC 101, MCC UF702, HPMC, cornstarch, erythritol and lubricants were selected and mixed with gabapentin at 1:3 weight ratio. The powder mixes were stored at 60° C. for short-term compatibility screening. After 1 and 2 weeks, powder mixes were analyzed by HPLC for assay and lactam levels. As exhibited in Table 5, lactam levels increased at accelerated condition, and the levels were above 10% in poloxamer 188, poloxamer 407, aerosol and talc. Among all the tested excipients, PVP, cornstarch and erythritol presented the best compatibility.

TABLE 4
Excipient-Gabapentin Compatibility

| | | Assay | Lactam |
|---|---|---|---|
| Excipient | Condition | (mg/g) | (%) |
| PVP-VA64 | 1 week @ 60° C. | 21.59 | 0.29 |
| | 2 weeks @ 60° C. | 20.93 | 0.31 |
| PVP-K30 | 1 week @ 60° C. | 21.09 | 0.06 |
| | 2 weeks @ 60° C. | 21.94 | 0.08 |
| Poloxamer 188 | 1 week @ 60° C. | 5.93 | 3.49 |
| | 2 weeks @ 60° C. | 3.63 | 18.00 |
| Poloxamer 407 | 1 week @ 60° C. | 2.51 | 1.82 |
| | 2 weeks @ 60° C. | 1.82 | 21.28 |
| MCC 101 | 1 week @ 60° C. | 22.44 | 0.38 |
| | 2 weeks @ 60° C. | 22.44 | 0.60 |
| HPC-SL | 1 week @ 60° C. | 19.5 | 1.39 |
| | 2 weeks @ 60° C. | 18.48 | 2.56 |
| HPMC | 1 week @ 60° C. | 16.95 | 0.39 |
| | 2 weeks @ 60° C. | 22.19 | 0.57 |
| Cornstarch | 1 week @ 60° C. | 24.31 | 0.20 |
| | 2 weeks @ 60° C. | 25.82 | 0.26 |
| Magnesium Stearate | 1 week @ 60° C. | 18.23 | 0.20 |
| | 2 weeks @ 60° C. | 25.49 | 3.28 |
| Talc | 1 week @ 60° C. | 23.65 | 0.93 |
| | 2 weeks @ 60° C. | 23.58 | 1.57 |
| Aerosol | 1 week @ 60° C. | 22.42 | 6.30 |
| | 2 weeks @ 60° C. | 21.10 | 11.19 |
| Erythritol | 1 week @ 60° C. | 25.61 | 0.07 |
| | 2 weeks @ 60° C. | 25.12 | 0.33 |
| MCC UF702 | 1 week @ 60° C. | 24.04 | 0.39 |
| | 2 weeks @ 60° C. | 22.65 | 0.75 |

### Example 4

### Excipient and Flavoring Options

Based on the excipient compatibility results, CMC-Na-7LF, PVP-K30, and erythritol were compared for moisture control, and improved wet granulation and dry powder mixing processes. A set of formulations listed in the Table 5 were prepared using wet granulation and dry powder mixing, individually.

TABLE 5
Formulation Screening

| | Content (%) | | | |
|---|---|---|---|---|
| Ingredients | 0507-P1 (M) | 0507-P2 (M) | 0507-P3 (M) | 0507-P4 (M) |
| Gabapentin | 20 | 20 | 20 | 20 |
| CMC-NA-7LF | 20 | 30 | — | — |
| PVP-K30 | — | 44 | — | 74 |
| Erythritol | 54 | — | 74 | — |
| Acesulfame | 3 | 3 | 3 | 3 |
| Potassium | | | | |
| Saccharin | 3 | 3 | 3 | 3 |
| Sodium | | | | |
| PBS (pH 6.8) | As needed | As needed | As needed | As needed |

Formulations were prepared by either wet granulation or direct powder mixing, and samples are stored at accelerated conditions for short-term stability screening. As presented in Table 9a and 9b. Formulations prepared by direct powder mixing showed lower lactam levels compared with the same formulations prepared by wet granulation due to less water introduction (Tables 6 and 7). Formulations containing CMC-Na and erythritol exhibited lower lactam levels among all the formulations. To evaluate reconstitution, 1.0 g powder formulation was weighed into a cup, and about 100 mL room temperature water was added into the cup under spatula stirring. The existence of CMC-Na still showed prolonged reconstitution; reconstitution time of formulations containing CMC-Na was all greater than 30 seconds.

TABLE 6
Wet Granulation Formulation Stability

| | | Assay | Purity | Lactam | Reconstitution |
|---|---|---|---|---|---|
| Formulation | Condition | (%) | (%) | (%) | Time (sec) |
| 0507-P1 | 1 week @ 60° C. | 17.51 | 99.70 | 0.30 | 35 |
| | 2 weeks @ 60° C. | 20.07 | 99.40 | 0.61 | |
| | 4 weeks @ 60° C. | 19.91 | 98.88 | 1.12 | |
| | 2 weeks @ | 19.73 | 99.93 | 0.07 | |
| | 40° C./RH75% | | | | |
| | 4 weeks @ | 19.31 | 99.87 | 0.17 | |
| | 40° C./RH75% | | | | |
| 0507-P2 | 1 week @ 60° C. | 15.41 | 99.02 | 0.98 | 60 |
| | 2 weeks @ 60° C. | 15.82 | 98.75 | 1.25 | |
| | 4 weeks @ 60° C. | 17.12 | 98.61 | 1.39 | |
| | 2 weeks @ | 13.62 | 99.17 | 0.83 | |
| | 40° C./RH75% | | | | |
| | 4 weeks @ | 14.14 | 98.36 | 1.64 | |
| | 40° C./RH75% | | | | |
| 0507-P3 | 1 week @ 60° C. | 18.98 | 99.30 | 0.70 | 20 |
| | 2 weeks @ 60° C. | 19.57 | 98.27 | 1.73 | |
| | 4 weeks @ 60° C. | 19.62 | 95.3 | 4.37 | |
| | 2 weeks @ | 19.38 | 99.88 | 0.12 | |
| | 40° C./RH75% | | | | |
| | 4 weeks @ | 19.63 | 99.72 | 0.28 | |
| | 40° C./RH75% | | | | |
| 0507-P4 | 1 week @ 60° C. | 17.51 | 99.00 | 1.00 | 45 |
| | 2 weeks @ 60° C. | 17.07 | 98.48 | 1.52 | |
| | 4 weeks @ 60° C. | 17.24 | — | — | |
| | 2 weeks @ | 15.60 | 98.57 | 1.43 | |
| | 40° C./RH75% | | | | |
| | 4 weeks @ | 15.19 | 96.15 | 3.85 | |
| | 40° C./RH75% | | | | |

TABLE 7
Direct Powder Mixing Formulation Stability

| | | Assay | Purity | Lactam | Reconstitution |
|---|---|---|---|---|---|
| Formulation | Condition | (%) | (%) | (%) | Time (sec) |
| 0507-P1-M | 1 week @ 60° C. | 17.49 | 99.94 | 0.06 | 50 |
| | 2 weeks @ 60° C. | 17.35 | 99.94 | 0.07 | |
| | 4 weeks @ 60° C. | 19.94 | 99.90 | 0.10 | |
| | 4 weeks @ | 17.61 | 99.88 | 0.12 | |
| | 40° C./RH75% | | | | |
| 0507-P2-M | 1 week @ 60° C. | 15.73 | 99.82 | 0.18 | 80 |
| | 2 weeks @ 60° C. | 18.92 | 99.79 | 0.21 | |
| | 4 weeks @ 60° C. | 16.23 | 98.91 | 1.09 | |
| | 4 weeks @ | 17.59 | 99.82 | 0.18 | |
| | 40° C./RH75% | | | | |
| 0507-P3-M | 1 week @ 60° C. | 18.10 | 99.90 | 0.10 | 20 |
| | 2 weeks @ 60° C. | 19.50 | 99.79 | 0.21 | |
| | 4 weeks @ 60° C. | 19.84 | 99.87 | 0.68 | |
| | 4 weeks @ | 19.55 | 99.32 | 0.13 | |
| | 40° C./RH75% | | | | |
| 0507-P4-M | 1 week @ 60° C. | 19.86 | 99.93 | 0.07 | 60 |
| | 2 weeks @ 60° C. | 17.49 | 99.85 | 0.15 | |
| | 4 weeks @ 60° C. | 19.54 | 99.19 | 0.81 | |
| | 4 weeks @ | 17.22 | 97.74 | 2.26 | |
| | 40° C./RH75% | | | | |

To obtain a both palatable and stable formulation, acesulfame potassium and saccharin sodium were selected as sweeteners based on a previous study of gabapentin solution. Acesulfame potassium and saccharin sodium exhibited better stability in gabapentin solution formulations compared with other sweeteners. Different flavors were also evaluated in the powder formulation. Gabapentin and erythritol were screened through a 300 μm sieve and mixed with desired amount of sweeteners and flavors. The formulations were put into glass vials with caps and then stored at 60° C., 40° C./75% RH, and 25° C./60% RH stability chambers for short-term stability screening.

After 2 and 4 weeks, formulations were sampled for purity and lactam level evaluation by HPLC. The lactam levels increased faster at 60° C. compared with samples at 40° C./75% RH. After 4 weeks, the formulation containing raspberry-bubble gum flavor showed excellent stability, with lactam levels reaching 0.14% and 0.65% at 40° C./75% RH and 60° C., respectively, which is comparable to or the formulation without any flavor (lactam levels of 0.13% and 1.06% at 40° C./75% RH and 60° C., separately). The formulation containing strawberry flavor presented acceptable lactam levels after 4 weeks at accelerated conditions.

### Example 5

### Palatability, Reconstitution Time, and Stability of Flavored Formulations

The palatability of the formulations from Example 4 were evaluated by taste testing. Formulation powder (1.5 gram) was weighed into a 240 mL cup and 100 mL of water was added with spoon stirring to dissolve the formulations. The palatability of different formulations was evaluated by about 10 people and the most palatable formulation was selected after tasting the reconstituted solution.

The water content of the prepared formulation was evaluated by Karl-Fischer titration. The water content of the fresh-prepared formulation was about 0.6%. To compare the necessity of drying process, the formulation was put into vacuum oven to dry overnight at 40° C. with the pressure of <0.1 mbar. The water content of formulation after drying was also tested by Karl-Fischer titration, and the water content level decreased to about 0.3%. Considering the low initial water content, vacuum drying is not necessary or included in final process.

The reconstitution time for the powder formulation in water was also evaluated. Powder formulation (1.5 g) was weighed into a 200 mL glass beaker and 100 mL room temperature water was poured into the beaker. A spatula was used to stir the mixture until the powder was completed dissolved. The powder formulation dissolved in water within 15 secs.

Powder formulation was put into glass bottles with caps and stored at 60° C., 40° C./75% RH, and 25° C./60% RH chambers for stability screening. After 0.5, 1, 2, 3, 4, 5, and 6 months, samples were pulled and evaluated by physical appearance, assay, impurities, and lactam levels (Table 8). Formulation stored at 40° C./75% RH and 25° C./60% RH chambers remained a flowable powder, however, the formulation stored at 60° C. showed aggregation after 1 month due to the high temperature. The lactam levels increased significantly at 60° C. and reached 0.96% after 4 weeks. The lactam levels stayed low at other storage conditions and it was only 0.1% after 6 months at 40° C./75% RH.

TABLE 8
Stability Evaluation of Gabapentin Powder Formulation

| | Assay | Purity | Lactam |
|---|---|---|---|
| Condition | (mg/g) | (%) | (%) |
| T0 (initial) | 201.8 | 99.98 | 0.02 |
| 2 weeks @ 40° C./RH75% | 203.1 | 99.96 | 0.04 |
| 2 weeks @ 60° C. | 201.2 | 99.39 | 0.61 |
| 4 weeks @ 40° C./RH75% | 200.0 | 99.89 | 0.11 |
| 4 weeks @ 60° C. | 198.0 | 99.04 | 0.96 |
| 12 weeks @ 40° C./RH75% | 201.4 | 99.92 | 0.08 |
| 4 months @ 40° C./RH75% | 196.9 | 99.92 | 0.08 |
| 4 months @ 25° C./RH60% | 190.2 | 99.97 | 0.03 |
| 5 months @ 40° C./RH75% | 202.8 | 99.90 | 0.10 |
| 6 months @ 40° C./RH75% | 213.6 | 99.90 | 0.10 |

### Example 6

### Process Development

Sieve Selection-To avoid ingredient agglomeration, gabapentin and excipients were screened through sieves before mixing. To obtain the suitable sieves for different excipients, the particle sizes for all the components were evaluated by microscope observation. The particle sizes follow the sequence: flavor <erythritol (˜100 mesh)<gabapentin (˜100 mesh)<acesulfame potassium <saccharin sodium. The flavor features the smallest size, and erythritol and gabapentin feature similar size, and all of them can easily pass through a 300 μm sieve. Due to the wide size distribution of acesulfame potassium, a 600 μm sieve is suggested.

Powder Mixing—To prepare a small-scale gabapentin powder mix formulation, a Turbula mixer was used. To obtain a suitable mixing time, a formulation containing 20% gabapentin, 20% CMC-Na, 54% Erythritol, 3% Acesulfame potassium and 3% saccharin sodium was prepared by Turbula direct mixing. After 5-, 10- and 20-min mixing, 6 samples were pulled for assay. The results show that 10 min mixing presented the lowest standard deviation, which was 100.25+1.47%.

Scale-Up—A 1.0 kg scale-up batch was prepared comprising 20% gabapentin, 75.5% erythritol, 4% sweeteners, and flavoring. To avoid any powder aggregation, gabapentin, erythritol and the flavoring were screened through 300 μm sieve and the sweeteners were screened through 600 μm sieves. All the ingredients were transferred into a V-blender and mixed for 15 min. Then the powder formulation was filled into stick packs manually targeting 1.50 g fill weight (1.455 g to 1.545 g, ±3%) and sealed using a heat sealer.

The scale-up batch was used to determine various physiochemical parameters. The yield of the 1.0 kg batch was 97.6%. The target formulation was a flowable white powder. The powder was easily reconstituted in room temperature water within 20 secs. The initial assay was 193 mg/g (290 mg/pack). The Carr's Index was tested to be roughly 31, but the angle of repose was tested to be 32 degrees, which exhibited a nice powder flowability. The bulk density was tested to be 0.58 g/mL and the tap density was 0.83 g/mL.

The scale up bath was also used to test stability of the stick packs at both 25° C./RH60% and 40° C./RH75% at 1, 3, and 6 months with additional analysis at 9, 12, and 18 months for 25° C./RH60% (Table 9). The lactam level after 6 months at 40° C./RH75% was higher than previous research batches which may be attributed to the increased permeability of the stick pack when compared with the glass vials. However, at room temperature the stability was comparable that seen with the glass vials and is stable for at least 18 months at 25° C./RH60%.

TABLE 9
Scale Up Formulation Stability

| | Assay | Purity | Lactam | Reconstitution |
|---|---|---|---|---|
| Condition | (mg/g) | (%) | (%) | Time (sec) |
| T0 (initial) | 193.3 | 100 | 0 | <20 |
| 1 month @ | 190.7 | 99.90 | 0.10 | <20 |
| 40° C./RH75% | | | | |
| 3 months @ | 193.1 | 99.96 | 0.04 | <20 |
| 25° C./RH60% | | | | |
| 3 months @ | 195.0 | 99.72 | 0.28 | <20 |
| 40° C./RH75% | | | | |
| 3 months @ RT | 194.1 | 99.97 | 0.03 | <20 |
| (bulk material) | | | | |
| 6 months @ | 176.0 | 98.61 | 1.39 | <20 |
| 40° C./RH75% | | | | |
| 6 months @ | 187.5 | 99.94 | 0.06 | <20 |
| 25° C./RH60% | | | | |
| 9 months @ | 201.9 | 99.91 | 0.09 | <20 |
| 25° C./RH60% | | | | |
| 12 months @ | 185.8 | 99.92 | 0.08 | <20 |
| 25° C./RH60% | | | | |
| 18 months @ | 197.2 | 99.84 | 0.16 | <20 |
| 25° C./RH60% | | | | |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, packaging, or methods of use of the invention, may be made without departing from the spirit and scope thereof.

What is claimed is:

1. A powder formulation of gabapentin comprising:
gabapentin;
a sugar alcohol; and
one or more artificial sweeteners,
wherein the formulation does not comprise any additional preservatives.

2. The powder formulation of claim 1, wherein the formulation comprises the gabapentin and the sugar alcohol at a weight percent ratio of 1:2 to 1:5.

3. The powder formulation of claim 1 or claim 2, wherein the formulation comprises the gabapentin and the one or more artificial sweeteners at a weight percent ratio of 1:0.01 to 1:0.5.

4. The powder formulation of any of claims 1-3, wherein the formulation comprises 10-30 weight percent gabapentin.

5. The powder formulation of any of claims 1-4, wherein the formulation comprises 60-80 weight percent of the sugar alcohol.

6. The powder formulation of any of claims 1-5, wherein the formulation comprises 0.2-10% of the one or more artificial sweeteners.

7. The powder formulation of any of claims 1-6, comprising:
10-30% by weight gabapentin;
60-80% by weight sugar alcohol; and
0.2-10% by weight one or more artificial sweeteners.

8. The powder formulation of any of claims 1-7, wherein the sugar alcohol is selected from the group consisting of: erythritol, sorbitol, xylitol, mannitol, galactitol, threitol, and combinations thereof.

9. The powder formulation of any of claims 1-8, wherein the sugar alcohol comprises erythritol.

10. The powder formulation of any of claims 1-9, wherein the one or more artificial sweeteners are selected from the group consisting of: aspartame, sucralose, neotame, acesulfame potassium, saccharin, advantame, and combinations thereof.

11. The powder formulation of any of claims 1-10, wherein the one or more artificial sweeteners comprise acesulfame potassium and saccharin sodium.

12. The powder formulation of any of claims 1-11, wherein the powder formulation further comprises a flavoring agent.

13. The powder formulation of claim 12, wherein the powder formulation comprises less than 1% by weight flavoring agent.

14. The powder formulation of any of claims 1-13, wherein gabapentin lactam impurities are below 0.05% following at last 3 months at room temperature.

15. The powder formulation of any of claims 1-14, wherein gabapentin lactam impurities are below 0.05% following at last 3 months at 25° C. and 60% relative humidity (RH).

16. The powder formulation of any of claims 1-15, wherein gabapentin lactam impurities are below 0.5% following 4 weeks at 40° C. and 75% relative humidity (RH).

17. The powder formulation of any of claims 1-16, wherein gabapentin lactam impurities are below 0.5% following 6 months at 40° C. and 75% relative humidity (RH).

18. The powder formulation of any of claims 1-17, wherein gabapentin lactam impurities are below 1% following 4 weeks at 60° C.

19. The powder formulation of any of claims 1-13, wherein gabapentin lactam impurities are below 0.2% following at least 12 months at 25° C. and 60% relative humidity (RH).

20. The powder formulation of claim 19, wherein gabapentin lactam impurities are below 0.2% following at least 18 months at 25° C. and 60% relative humidity (RH).

21. The powder formulation of any of claims 1-20, wherein the powder formulation exhibits a dissolution time of less than 1 min when dissolved at 1-2% weight to volume with mixing at room temperature.

22. The powder formulation of any of claims 1-20, wherein the powder formulation exhibits a dissolution time of less than 30 seconds when dissolved at 1-2% weight to volume water with mixing at room temperature.

23. A unit dosage comprising the powder formulation of any of claims 1-22.

24. The unit dosage of claim 23, wherein the unit dosage is a stick pack or sachet.

25. A method of preparing the powder formulation of any of claims 1-22, comprising direct powder mixing of the gabapentin, the sugar alcohol, and the one or more artificial sweeteners.

26. A method of treating or preventing a disease or disorder in a subjection comprising administering an effective amount of the powder formulation of any of claims 1-22 to the subject.

27. The method of claim 26, wherein the administering comprises oral administration.

28. The method of claim 26 or 27, wherein the administering comprises enteral feeding.

29. The method of any of claims 26-28, wherein the method comprises dissolving the powder formulation in a volume of water prior to administration.

30. The method of any of claims 26-29, wherein the disease or disorder comprises a neurological or central nervous system disease or condition.

31. The method of any of claims 26-30, wherein the disease or disorder comprises seizures, neuropathic pain, chronic pain, neurodegenerative disorders, hypokinesia, and bradykinesia.

32. Use of powder formulation of any of claims 1-22 for the manufacture of a medicament for the treatment of a disease or disorder.

33. The use of claim 32, wherein the disease or disorder comprises a neurological or central nervous system disease or condition.

34. The use of any of claim 32 or claim 33, wherein the disease or disorder comprises seizures, neuropathic pain, or chronic pain.
